(19) United States
(12) Patent Application Publication
(10) Pub. No.: US 2022/0054377 A1
(43) Pub. Date: Feb. 24, 2022

(54) CHEMICAL PROCESS TO TRANSFORM BODY ODOR

(71) Applicant: Aidan William Oster, New Hope, PA (US)

(72) Inventor: Aidan William Oster, New Hope, PA (US)

(21) Appl. No.: 16/998,806

(22) Filed: Aug. 20, 2020

(51) Int. Cl. A61K 8/34; A61K 8/25; A61K 8/99; A61K 8/37; A61K 8/891; A61Q 15/00

(52) U.S. Cl. CPC A61K 8/342; A61K 8/345; A61K 8/25; A61K 8/99; A61K 2800/59; A61K 8/891; A61Q 15/00; A61K 2800/48; A61K 2800/87; A61K 8/37

(57) ABSTRACT

Compositions for incorporation into a deodorant or antiperspirant product for reducing, preventing, or transforming perspiration and odor associated with perspiration. Compositions for initiating a reaction between a catalyst and acid present in perspiration generated on the body of a human subject.

### BACKGROUND

The instant disclosure is in the field of personal care, particularly personal care compositions for reducing, transforming or preventing unwanted perspiration and odor associated with perspiration. More specifically, the instant disclosure pertains to compositions for use in a deodorant and antiperspirant product for application to the skin of a human subject.

Sweating is the release of a salty liquid from the sweat glands. The liquid has one main purpose: as it evaporates, it helps to cool the body. Sweating is regulated by the autonomic, or sympathetic nervous system. Signals, using the neurotransmitter acetylcholine, are sent to the sweat glands. The sweat is then released to the skin surface through ducts.

Body odor generally refers to a condition in which an unpleasant smell or a malodor is released from a body part or skin of a body part of a human subject. Although body odor is known to be influenced by various physiological conditions, it is generally related to perspiration or sweating of the subject, and the subsequent bacterial decomposition of the organic compounds present in the sweat which forms the malodor carrying volatile organic acids. It is therefore most common for body odor to develop at axillary regions of the body such as the underarm areas where a large number of sweat glands are located, although body odor may also develop at other parts of the human body such as the palms of the hands, the soles of the feet, the upper thighs and the back region.

Odors from the apocrine glands under the arms (e.g., armpits) are typically produced when a person sweats and when bacteria have started to develop on the surface of the body. However, the odor is not from sweat alone, but from the product of bacteria metabolizing proteins, carbohydrates, or starches in sweat. The product, and the cause of the odor, is typically propionic acid and isovaleric acid. Deodorants and antiperspirants are thus made for application in the sweat areas and to create a harsher environment for the bacteria to exist. In spite of the application of most deodorants, however, bacteria still exist or thrive and a malodor may still be present.

Present methods of preventing or removing odor include the use of antiperspirants and deodorants which attempt to kill the live bacteria that cause smell or to prevent the bacteria from growing. As noted, body odor is caused by the bacteria that is already present on the body metabolizing the compounds in sweat. The bacteria metabolize these compounds into acids, which are the cause of odor. Deodorants inhibit the odor causing bacteria that make your sweat smell. On the contrary, antiperspirants are designed to reduce sweating, while also inhibiting/reducing sweat odor.

Various deodorant compositions and/or preparations have been developed to reduce, alleviate, eliminate or mask the unpleasant smell, and/or to suppress or inhibit the production of sweat from the skin of the body parts. Deodorants may work by providing an antiseptic effect to inhibit the bacterial decomposition; by incorporating an anti-perspiring agent to suppress or inhibit perspiration; and/or by introducing fragrance to mask the generated odor. Deodorant preparations may come in different forms, such as solid, gel, cream, liquid or aerosol for various applications such as apply-on or roll-on sticks or rollers, body sprays or even body wipes; and can be classified as a cosmetic or a pharmaceutical product depending on the specific active ingredients in the composition

Deodorants also work by creating a dry environment, which makes it harder for the bacteria to live and grow and thus eliminate the bacteria responsible for metabolizing the materials. The compounds that are used to kill bacteria usually cannot target certain bacteria. Therefore, the antibacterial compounds kill both bad bacteria and the good bacteria that exist on a body.

Deodorants are typically formulated to eliminate armpit odor but not perspiration. They are typically alcohol-based, and when applied, they turn your skin acidic, which makes it less attractive to bacteria. Deodorants also commonly contain perfume to mask odor.

Antiperspirant deodorants work to maintain a dry environment in the area to which it is applied. The antiperspirants work by covering the sweat ducts that emit sweat. The active ingredients in antiperspirants usually include aluminum-based compounds that temporarily block sweat pores. Blocking sweat pores reduces the amount of perspiration that reaches your skin.

Unfortunately, metal-containing compounds such as aluminum salts that are widely used for their ability to reduce perspiration have been the subject of as-yet unresolved concerns about their long-term health effects. Some people are allergic to aluminum and may suffer contact dermatitis after using aluminum-containing antiperspirants. Some reports have linked aluminum-containing antiperspirants with the systemic accumulation of aluminum in the body, which may be a risk factor for Alzheimer's disease or breast cancer. See, for example, C. Exley, “Does Antiperspirant Use Increase the Risk of Aluminum-Related Disease, Including Alzheimer's Disease?,” Molecular Medicine Today 4 (3): 107-9 (March 1998); K. G. McGrath K G, “An Earlier Age of Breast Cancer Diagnosis Related to More Frequent Use of Antiperspirants/Deodorants and Underarm Shaving,” European Journal of Cancer Prevention, 12 (6): 479-85 (December 2003); and P. D. Darbre, “Underarm Antiperspirants/Deodorants and Breast Cancer,” Breast Cancer Research 11 (Suppl 3):S5 (2009).

The instant disclosure contemplates a composition for eliminating the negative smell or malodor generated within one's body sweat and perspiration. A composition is contemplated that can be incorporated into deodorants and antiperspirants that exist in the form of solid sticks, roll-ons, gels, wet wipes, soaps, and sprays. Spray embodiments typically include aerosols, natural sprays and squeeze sprays.

The instant disclosure also contemplates compositions that can be incorporated into laundry detergent, air fresheners, car fresheners and clothing sprays to eliminate or reduce odors. The compositions will facilitate the reaction of organic acids into esters.

The contemplated deodorants and related products of the instant disclosure contain ingredients that transform the acids that produce malodor and make them or their smell weaker or transform them into an ester or other compound that produces a pleasant smell. In this manner, the acid exists almost as a live and on-demand source or fuel to create the pleasant smell in this contemplated deodorant and antiperspirant.

Given the limitations in currently available products, there is a need for deodorants and antiperspirant products that are effective in transforming perspiration and associated malodor on the body, while reducing perceived health risks associated with aluminum compounds or other harsh chemicals.

### SUMMARY

The instant disclosure contemplates a novel composition for use in reducing, inhibiting or suppressing development of malodor from a body part of a human subject by transforming and thereby removing the source of smell and that substantially reduces, and in many cases eliminates, negative body odor that results from perspiration.

As noted hereinabove, improving body odor is a large part of hygiene, and which includes a substantial consumer market. Deodorants and antiperspirants are widely used consumer products and companies that sell these products continually research new technologies to create an efficient way to avoid unpleasant body odor. Deodorants work to create an environment in the applied area that makes it hard for bacteria to survive.

Antiperspirants attempt to inhibit perspiration in an applied area and often contain deleterious ingredients. Potentially harmful chemicals in some antiperspirants and deodorants may, however, detract from their widespread use. Many deodorants and antiperspirants are also simply weak and overpowered by perspiration or the body's production of odor, and are ineffective, effective for a short duration, or effective when perspiration is minimal.

In addition to deodorants and antiperspirants, other products may be applied to areas of perspiration to add a pleasant smell, e.g., perfume, essential oils and powders.

Bacteria that are present on one's body, particularly in the armpits, metabolize the proteins that exist in sweat. These bacteria produce organic acids, which cause the unpleasant smell. Examples of bacteria that produce these organic acids that result in malodor include, but are not limited to, Propionibacterium acnes, Staphylococcus epidermis, brevi bacterium spp., Bacillus subtilis, Staphylococcus aureus, micrococcus spp, and others. The organic acids typically produced by these bacteria include, but are not limited to, acetic acid, propionic acid, isobutyric acid, butyric acid, valeric acid, isovaleric acid, caproic acid, caprylic acid and capric acid. Isovaleric acid is a well-known cause of disagreeable smell that results in unpleasant body odor.

Starches, carbohydrates, or proteins facilitate the process of organic acid metabolization by the bacteria by providing the bacteria with ingredients that they need to metabolize and live. However, providing the bacteria with specific starches, carbohydrates, or proteins can facilitate metabolization such that the bacteria will not create any odor. Specific sugars, such as glucose, can also be used and which do not produce a smell when metabolized. Certain starches, proteins, carbohydrates, and sugars are often sticky, and thus the starch, carbohydrate or protein used in the instant composition will be selected from those that do not exbibit smelly or sticky properties.

The reaction of an alcohol and an acid produces the acid's ester with that alcohol and water. This reaction is known as esterification and these reactions reach equilibrium.

The esterification reaction is particularly useful in the food industry and personal hygiene industry. There are many studies that describe how to make this reaction produce high levels of ester quickly and efficiently. See, e.g, Chemical Engineering Science, Volume 62, Issue 12, June 2007, Pages 3197-3217; Industrial & Engineering Chemistry Research 48(5), February 2009

Many ethyl esters, such as ethyl isovalerate are used to flavor food and create a pleasant odor in perfumery and deodorants. Esters also have antibacterial properties and are thus often used in deodorants. However, the reaction of creating an ester is usually slow without a catalyst. To make this reaction more efficient, catalysts, enzymes, and other factors are used to accelerate the reaction.

The ethyl esters of the exemplary organic acids typically have pleasant smells and, in some cases, a neutral smell or no smell at all. Acetic acid's ethyl ester is ethyl acetate and does not have an unpleasant smell. Propionic acid's ethyl ester is ethyl propionate and has the smell of pineapple. Isobutyric acid's ethyl ester is the ethyl isobutyrate and has the smell of oranges and grapefruit. Butyric acid's ethyl ester is ethyl butyrate, which has the smell of orange juice. Valerie acid's ethyl ester is ethyl valerate and is used in cosmetics for its pleasant smell. Isovaleric acid's ethyl ester is ethyl isovalerate and has the smell of apples. Caproic acid's ethyl ester is ethyl caproate and has a pleasant smell. Caprylic acid's ethyl ester is ethyl caprylate and is used in perfumery. Capric acid's ethyl ester is ethyl caprate and has a pleasant smell that is used in beverages.

When a substantial amount of water is present, the desired esterification reaction reverses. The water reacts with the ester and makes the alcohol and the acid. This is called hydrolysis, and this is not optimal for the reaction of the alcohol and the ester. To ensure that the reaction produces ester and not alcohol, the reaction conditions need to favor making the ester and not the alcohol.

As noted, esters of the organic acids mentioned above are known to be excellent ingredients for flavoring and producing pleasant smells in hygiene products. These esters of the organic acids are ideal for being a product of the esterification reaction, as many of these acids are found in small amounts and can react with the alcohol. A suitable catalyst for the esterification reaction will catalyze the esterification reaction of any acid.

### DETAILED DESCRIPTION

All percentages expressed herein are by weight unless otherwise indicated.

As used herein, “deodorants” and “antiperspirants” both refer to compositions that are effective in directly or indirectly reducing unwanted body odors associated with perspiration and/or bacteria on the surface of the skin. “Deodorants” may reduce odor through a variety of means, and such means in the various embodiments of the present invention may include suppression of bacterial activity, antimicrobial mechanisms, chemical interference with odor generation mechanisms, removal or modification of feedstuff for odor-producing bacteria, and the like. “Antiperspirants” generally work to reduce the production of perspiration, and may do so, for example, by decreasing the size of pores associated with sweat glands, blocking sweat glands, reducing the flow of blood or other fluids to sweat glands, and the like.

The terms “smell”, “odor”, “aroma”, and “scent” are used herein throughout in an interchangeable manner and are all intended to connote the general definition to describe the smell of perspiration and sweat.

To create a deodorant or antiperspirant product in accordance with the instant disclosure, typically in the form of a solid stick, gel, cream, liquid or wet wipe, roll-on, or a spray, that produces esters from organic acids and thus are capable of masking or transforming the unpleasant smell of perspiration, the deodorant composition will incorporate ingredients that undergo reactions with bacteria that exist on the human body and which, in turn, generate a malodor or unpleasant smell.

In one embodiment, such a reaction is an esterification reaction. Esterification reactions combine organic acids and alcohols making esters and water. This reaction is particularly useful to the food industry and personal hygiene industry. There are many studies that describe how to make this reaction produce high levels of ester quickly and efficiently. Many ethyl esters such as ethyl isovalerate are used to flavor food and create a pleasant odor in perfumery and deodorants. Esters also have antibacterial properties and are thus used in deodorants. However, the reaction of creating an ester is usually slow without a catalyst. To make this reaction more efficient, catalysts, enzymes, and other factors are used to accelerate the reaction.

The general esterification reaction contemplated in the instant disclosure is: EtOH+RCOOH<-->EtOCR+H₂O, where Et is ethyl. EtOH is ethanol. R is any organic group. Ethanol is used as a model; however, many alternative alcohols can be used and would create a similar reaction.

As noted, the general esterification process reacts an alcohol with an acid. In certain embodiments, alcohols are used in the mixture to create esters. Alcohols with 1 to 29 carbons are preferable since they typically generate pleasant smells.

In certain embodiments, monoterpene alcohols generate favorable pleasant smells.

In certain embodiments, the instant composition includes esters of the alcohols that generate favorable pleasant smells.

In certain embodiments, esters and alcohols may be incorporated into the composition to react with the acids. This reaction typically will not produce any smell. The acids used in this embodiment will typically comprise 12 to 34 carbons. Acids with this number of carbons typically do not have a strong odor and will be less reactive than isovaleric acid or another acid, therefore, the ester of isovaleric acid can be produced. If an ester is used in the composition, it should be combined with an acid with this number of carbons since it typically will not generate a smell, and the reaction will be faster if the acid has this number of carbons.

In certain embodiments, a composition comprising an ester and a strong anion exchange resin can be incorporated in a deodorant or antiperspirant formulation. The ester will react with the water produced and create the acid and the alcohol. The strong anion exchange resin will act as a base and create a salt with the acid. Glycerol esters have shown efficacy because they usually have antibacterial properties. Glycerol esters also have three acids for one glycerol. Therefore, if an antibacterial acid is used, there will be a large amount of this acid that will not cause bacteria to make a large volume of isovaleric acid.

If a base is used, such as, for example, zinc hydroxide, the acid will react to make the zinc salt, or the salt of the metal that is used in the base. These salts are antibacterial and will cause the area to have less bacteria.

In certain embodiments, the present technology contemplates a mixture of an enzyme or catalyst and an alcohol in order to speed the reaction and increase ester yield. Some exemplary suitable enzymes for this reaction are Novozyme 435, Lipozyme CALB L, Novozyme TL IM, Novozyme Eversa liquid lipase, Novozyme IM 20. These products are manufactured by Novozymes Corp.

These Novozyme enzymes are lipases that can catalyze the esterification of isovaleric acid and other organic acids. The lipases or enzymes come from bacteria, e.g. Candida rugosa, Candida antarctica, Aspergillus niger, Aspergillus oryzae, Thermomyces lanuginosus, and Rhizomucor miehei. Studies involving these enzymes have shown that they do catalyze the esterification of isovaleric acid, butyric acid, and other acids. (See, e.g., Appl Biochem Biotechnol. 2015; 175(5): 2769-2785; Biocatalysis and Agricultural Biotechnology, Volume 1, Issue 1, January 2012, Pages 51-56; Int J Mol Sci. 2012; 13(9): 11694-11704. Many of these lipase enzymes comprise another category of enzymes known as hydrolases.

Hydrolases are meant to catalyze reactions that have water as a reactant (i.e. hydrolysis). Lipases can also catalyze the esterification reaction and they catalyze the esterification most effectively when there is no water in the applied area. As such, these lipase enzymes can be useful in the instant embodiments based on their ability to catalyze esterification reactions.

In certain embodiments, polyester synthase enzymes may also be used.

Acyltransferases comprise another category of enzymes that have the ability to catalyze esterification reactions in certain embodiments.

Acyltransferase from Mycobacterum smegmatis can catalyze the production of esters. Another acyltransferase, alcohol acyltransferase, is known to catalyze esters and these enzymes have been found in plants and flowers. Alcohol acyltransferase is found in fruits and is the enzyme that produces the esters in fruits. In fact, ethyl isovalerate and many esters are found in fruits. Some of these esters will be produced in the esterification reaction of the organic acids that cause body odor and are created by bacteria. Therefore, alcohol acyltransferases can be used to catalyze the esterification of isovaleric acid and the other organic acids that cause body odor.

Two specific groups of acyltransferases are useful to catalyze these reactions. These groups are BAHD acyltransferases and SCPL acyltransferases. BAHD-acyltransferases, named after the first four biochemically characterized enzymes of the group, use acyl-CoA thioesters as donor molecules, whereas SCPL (Serine CarboxyPeptidase Like)-acyltransferases use 1-O-β-glucose esters.

In certain embodiments, a procedure for increasing the efficiency of the enzymes, and in turn, the catalyst/esterification reaction, involves immobilization of the enzyme(s) on objects or gels, and in certain embodiments they can be coated with one or more surfactants. The use of coating enzymes immobilized in microemulsion-based organogel (MBG), was studied for making ethyl isovalerate. Candida rugosa lipase was coated with several surfactants and immobilized in an MBG. Mixtures of the surfactant used to coat the enzyme and the surfactant was used in the preparing of the MBG. Of these mixtures, the best mixture was shown to be CTAB (cetyl triethylammonium bromide) coated enzyme immobilized in a di-sodium ethyl hexyl sulfosuccinate based MBG, which had an eight-fold higher production of ester than free lipase. Also, the enzyme could be immobilized on silica nanoparticles or graphene oxide particles, however, graphene oxide is not soluble in water and would be very hard to wash from the applied area, so it would not be the best object on which to be immobilized. The enzyme or lipase can also be immobilized on objects that have a high surface area or on objects that are hydrophilic, to increase efficiency. (See., e.g., Chemical Engineering Journal, Volume 334, 15 Feb. 2018, Pages 760-767)

In certain embodiments, the enzymes can be prepared by mixing them as unprepared enzymes, or they can be prepared by being immobilized on a support and mixed with a surfactant as noted above.

As noted, the esterification reaction is an equilibrium reaction. Reducing the amount of water will increase the ester yield. This hydrolysis reaction produces acid and alcohol. To facilitate this reaction (e.g., the production of ester), in certain embodiments, enzymes can be immobilized on a hydrophilic object, so water that would react with the ester and be catalyzed by the enzyme if the enzyme also catalyzes hydrolysis, would react with the hydrophilic object. An efficient object for this is gelatin. Other hydrophilic objects can be used, and hydrophilic objects that do not have lipases can also be present in a deodorant formulation or spray mixture, so the applied area is not moist. The CTAB surfactant coating and disodium ethyl hexyl sulfosuccinate MBG mentioned can also be treated with dry reverse micelle solution to make the applied area dry. This method is efficient, as demonstrated by the fact that it can be reused for several esterification reactions. This means that it can be efficient for the time that the deodorant or spray is applied, which could be a full day. Also, the immobilized enzyme or free lipase can be in a hydrophobic nanoporous shell. This allows the alcohol and acid to access the enzyme but does not allow the water to use the enzyme.

Since esterification has a dynamic equilibrium, preventing water from reacting with the ester will create the desired system. Therefore, when the water reacts with another compound or chemical, the back reaction is prevented and will produce the desired ester.

The reaction of the ester and water is not spontaneous, so if water cannot react with the enzyme, then the enzyme will catalyze the esterification, which will occur and produce a substantial yield of esters. These methods can be used for the lipases mentioned above because they also catalyze the hydrolysis reaction. If there is water in the applied area, the lipases favor undergoing the hydrolysis. Therefore, they will be most effective in catalyzing the esterification reaction if the area is dry. However, these methods are unnecessary when using the acyltransferases mentioned above because they favor undergoing the esterification. Although these methods increase the production of ester, the free lipases and free enzymes can also be used. While the immobilized enzyme does not need to be coated, coating the enzyme may increase the enzyme's efficiency.

Silica gel and hydrogel as beads or ground up particles can be used to coat the immobilized enzyme. Gelatin microemulsion-based organogels (MBG) may also be used. These materials can be used to immobilize the enzyme, and they can be present to dry the area. Therefore, the reaction will create ester and the ester will not react with the water. The reaction is a dynamic equilibrium, so if the hydrolysis reaction is not progressing quickly, then the reaction will produce a large volume of ester. While the silica gel and hydrogel can be used to dry the area, however, other desiccants can also be used.

Although water in the area reduces the amount of ester produced, water can be used as a solvent. The way to make this into the composition is to mix the water with silica gel. This water should not react with the esters and can therefore become a low-cost solvent. Using alcohols as solvents is more effective, but water can alternatively be used. Other desiccants can also work.

A molecular sieve can be used to dry the applied area, however, there is a possibility that the acid could react with the sieve and the esterification would not occur effectively. To alleviate this, the molecular sieve can be coated or reacted with a material that does not react with the acid. Additionally, the silica gel, hydrogel, or molecular sieve should not dry the alcohol. Several alternative drying agents can be used and are known by those having ordinary skill in the art.

In another embodiment, an ester can be used. Acidolysis in the case of esters, is the reaction of an ester and an acid to produce the ester of that acid.

Linalyl cinnamate is an exemplary ester that has a floral odor. The acidolysis reaction will produce linalyl isovalerate and cinnamic acid. The catalyst that should be used for this reaction is a heterogeneous acid catalyst. This catalyst will catalyze the reaction of the ester and water to produce an alcohol and an acid. This is a hydrolysis process. This catalyst also catalyzes the acidolysis reaction. The catalyst will also catalyze the reaction of the produced alcohol or alcohols and the isovaleric acid. The reaction of the ester and the isovaleric acid will not create a large volume of ester, so there will be unpleasant smelling acid in the area. However, esters will hydrolyze to produce the alcohol and the acid. Therefore, the acidolysis reaction will react and the alcohol and the acid will be produced. Acids with more carbons should be used because smaller acids produce esters faster. The alcohol will then react with the isovaleric acid to create an ester. This reaction will also produce water, and to help the esterification reaction, polyhydric alcohols can be used. Polycarboxylic acids can also be used for the esters in the deodorant.

Another ester that can be used is glyceryl laurate. This ester does not have a strong odor, but the reaction will make glyceryl isovalerate and stearic acid. The isovaleric acid ester does not have an odor. Linalyl pentadecanoate can also be used. This ester has a good odor. The ester it produces also has a pleasant odor and the acid does not have a strong odor. Catalysts may be added to facilitate the esterification reaction.

In certain embodiments that utilize an ester, a desiccant can also be used because if there is water, then the ester will produce the alcohol and the acid. This reaction has an equilibrium and might not produce a large volume of the ester. Therefore, this reaction can be used with an alcohol.

In the acidolysis composition, the ester should be made with an alcohol that makes a pleasant-smelling ester. The acid should have a high number of carbons, so the reaction is fast and it does not have a smell. The reaction typically does not need a catalyst. Therefore, the ester will not hydrolyze. However, if a catalyst is used, it should also catalyze the esterification. A cation exchange resin will catalyze the acidolysis. It will catalyze the hydrolysis. It will also catalyze the esterification. The hydrolysis will make an alcohol and an acid. The alcohol can make an ester with isovaleric acid. The reaction will make an ester and an acid. The reaction will use acid and water. Therefore, the reaction will give the area a pleasant smell and it can make the area less wet, or dry. The ester can also be used with a buffer or a hydrolase. This makes the ester produce an alcohol and an acid and will also dry the application area. The alcohol can be antibacterial, so the bacteria do not produce a large volume of acid. Glycerol and propylene glycol can also be used. The acid can also be antibacterial. Lauric acid is one such example. A polyhydric alcohol can be used, so a larger amount of water will react. An acid with many hydroxy groups, such as for example, citric acid can be used.

In another embodiment, one or more bacteria or fungi may be used as an enzyme to catalyze the esterification reaction. The bacteria or fungi that use the enzymes that catalyze the reaction of the ester, and the water can use the methods mentioned to produce a large volume of ester. There are good things about using bacteria. The bacteria that produce body odor have a population that increases, therefore, the production speed of isovaleric acid would also increase. If bacteria is used to catalyze the esterification and a large amount of alcohol is present, which will be discussed, then the bacteria population would also increase. In this case, the bacteria that cause odor will produce isovaleric acid at a high rate and the bacteria will also produce ester at a very high rate.

Lipases from the bacteria or fungi can catalyze the esterification reaction. The bacteria can survive in the conditions of an area that the deodorant or antiperspirant would work. However, the fungi could create infections and irritation, so they should be selected and used with discretion. The bacteria and fungi have the enzymes to catalyze the esterification, so they could also be used in the invention. As stated, the bacteria mentioned that have the lipases can catalyze the esterification. Additionally, the bacteria Pseudomonas fragi can be used. This bacterium can survive in the conditions of the applied area. This bacterium can also catalyze esterification reactions if an alcohol is present. Pseudomonas fragi does not cause infections. Pseudomonas fragi can produce several esters when alcohol and organic acids are present. An ester that was identified was ethyl butyrate, which is an ester of butyric acid that is one of the organic acids that cause odor. However, Pseudomonas fragi needs oxygen, which would make a product with the bacteria difficult or expensive to utilize. However, the product can use a porous container of bacteria wipes or stick applicator. Pseudomonas fragi also lives in low temperatures, so the area to which the deodorant or spray would be applied has suboptimal conditions for that bacteria given that the applied area will be the temperature of a person's skin.

Marinobacter hydrocarbonoclausticus is another bacterium that has demonstrated an ability to catalyze esterification reactions. This bacterium has a preference of catalyzing ethyl ester reactions. This bacterium does not need oxygen. However, it lives in salt water, so the bacteria would need to be applied in a saltwater solution. Any bacteria with the enzymes mentioned can be used to catalyze the reaction of making esters. If a long chain alcohol is used within the instant, then the enzyme or a bacterium with the enzyme long chain alcohol acyltransferase are candidates. However, the use of bacteria to catalyze the esterification is difficult because they may need oxygen and a certain temperature to live, which the packaging and applied area would need to accommodate.

As discussed, esterification reactions are usually catalyzed by acids or bases. However, while acids or bases may efficiently catalyze the reaction, certain acids or bases may irritate the applied area of the deodorant or spray. If the applied area is treated with a protective chemical or the acid or base is weak and does not irritate, it is suitable for use in the composition of the instant disclosure. Sulfuric acid, which has other safety issues, has been shown to be a suitable catalyst of esterification reactions.

Other suitable catalysts include strong cation exchange resins and strong anion exchange resins. These materials act as acids and bases that can catalyze the reaction, and they can be used as a powder. This can then be mixed with the composition of the alcohols or other ingredients. The strong cation exchange resin can be used in the acid or hydronium form, and the strong anion exchange resin can be used in the base or hydroxide form. However, the other forms of the exchange resins can be used. Some examples of known strong cation exchange that can be used include, but are not limited to, Amberlyst® 15, Amberlyst™ 35, Amberlyst™ 39, HZSM-5, Indion 170, Amberlyst IRA 120, Amberlyst™ 36, Amberlite IRA 120, Nafion silica composite acid catalyst, mesoporous sulphonated carbon catalyst, Amberlyst 70, Amberlyst BD 20 and Dowex H+ cation exchange resin. Of these, Amberlyst® 15, Amberlyst® 36, Nafion silica composite acid catalyst, and Dowex H+ cation exchange resin is the most effective for the esterification reaction. The Dowex H+ cation exchange resin can be dried and used with or without NaI or NaBr. NaI was used as a catalyst for the esterification of several organic acids and was highly effective.

An example of a strong anion exchange resin that catalyzes the reaction of making an ester is Purolite A 500. Other strong anion exchange resins can be used. Macroporous catalysts and heterogeneous catalysts are very good catalysts for esterification. These catalysts can be acidic. These catalysts can also be basic. Macroporous polymeric acid catalysts can also be used and can catalyze the esterification reaction with water in the area. Desiccants may also be used if needed.

Ferric alginate is a heterogeneous catalyst that is highly effective in catalyzing the esterification reaction. This can be irritating. However, other heterogeneous catalysts can also be used. Inorganic solids with an added hydroxyl group or a sulphonic acid functionalized silane can be used as a heterogeneous catalyst. M phenolsulfonic acid formaldehyde resin is an example of an acid resin that can be used, and other acid resins can be used. This type of catalyst was able to perform esterification reactions with the alcohol and acid entering, and the resulting reaction proceeds effectively. Although this does help because the acid will enter and be produced in the applied area, it is not required. The heterogeneous catalyst is usually used as beads, but it can be ground up and put it in a product. This catalyst can perform very effectively with water in the area; however, a desiccant can still be used. Any catalyst that can catalyze esterification can be used. Some of these catalysts could be irritating with types of bases or metals, so these chemicals should not be used with the catalysts for protection.

Graphene oxide is another heterogeneous catalyst that is effective in catalyzing esterification reactions.

In another embodiment, a catalyst that can be used and applied is a deodorant stick or object that can create heat or electricity to catalyze the esterification. Since a minimal amount of low voltage electricity or minimal heat is needed to catalyze the reaction, it would not damage the applied skin area. A battery or object that can create heat can be used within a deodorant stick. The alcohol or chemical used to make the ester would be applied and when there is isovaleric acid that has been produced, heat or electricity will be used to catalyze the reaction. The heat or electricity object will be the catalyst.

Many alcohols can be used in the esterification reaction. Most small alcohols make esters with strong fruity smells. Esters of alcohols with low molecular weight are usually used in perfumes and have fruity odors. Ethanol is an alcohol that is not toxic or irritating, and which does not have a strong odor, that can used because it creates ethyl isovalerate, which has a fruity smell. Other alcohols with low molecular weights can be used. Ethanol does not have a strong odor. Butanol, pentanol and hexanol are other examples of alcohols that can be used. These alcohols do not have strong unpleasant odors and create esters with pleasant smells.

Primary, secondary, and tertiary alcohols with low molecular weights can be used. Alcohols with high boiling points are the best. Tert-butanol is an alcohol that has a high boiling point and creates a pleasant-smelling ester, tert-butyl isovalerate. Alcohols with low molecular weights and high boiling points are the best because they will not evaporate, and they make esters with pleasant smells. However, if a fruity smell is not preferable, then alcohols with high molecular weights usually do not produce esters that have fruity smells.

Alcohols for producing an ester with no smell include those alcohols that can react to produce esters quickly. Since catalysts, enzymes or bacteria are being used, any alcohol can typically be used. If the deodorant or antiperspirant is used to create an environment with no odor, then alcohols with high molecular weights are optimal. Nonadecanol and undecanol can be used because they can produce esters that do not have strong odors. Alcohols with low boiling points can be made into powders so that as water transforms them into ethanol, the isovaleric acid will be produced and the ethanol can be used to create the ester. This can work with any alcohol. If a roll-on deodorant is being used, a solid alcohol can be used as well. Steranol is an alcohol that consists of white flakes at room temperature and makes esters that do not have strong odors. If an alcohol with low molecular weight is used with molecular sieves as a desiccant, then the 3 A molecular sieve should be used because this size will dry the water but not the alcohol.

Sesquiterpene and monoterpene alcohols can be used. Monoterpene alcohols have floral smells and make esters that have floral smells. Terpenol can be used because it has a floral smell and makes a pleasant-smelling ester. Perillyl alcohol can be used because it has the smell of many types of flowers and makes a pleasant-smelling ester. Many monoterpene alcohols can be used. These alcohols produce a pleasant smell. Sterols, which are waxes, can be used and sterol esters do not have substantial smell. Waxes, fats, and sugars can be used. Glycerol can be used because it is anti-bacterial, not toxic, and can make an ester with several organic acid molecules, so it will have a high molecular weight and will not smell.

To cause the esterification process to produce a large amount of ester, an excess of alcohol can be used. This will make the equilibrium favor making the ester. A highly concentrated solution of the alcohol can be used to produce a large volume of ester. If there is not a lot of alcohol, the equilibrium will favor making the acid.

For a spray formulation, any propellant can be used. Propanol can be used. The enzyme, catalyst or bacteria will be with the alcohol in the container. The bacteria can be dried and used with a solid alcohol. A mixture of several alcohols can be used. A mixture of an alcohol with another chemical that has a higher boiling point can be formulated. This will make an azeotrope with the alcohol and chemical, so the alcohol can make the ester and have a higher boiling point. In the deodorant, which can be a roll-on with a ball, spray or wet wipe, water is usually used for smooth application. However, an oil can be used, or several oils can be used. Santalol and linalool can be used, and these are alcohols that are oils. These make esters that have pleasant smells and they can be used to make the deodorant roll more easily. These oil alcohols are suitable because they will not be wet. For sprays, propellants are also used. Suitable propellants include a hydrocarbon or alcohol with 1 to 12 carbons. Hydrocarbon or alcohol with 3 to 8 carbons are better and 4-6 carbons are even better.

Carboxylic acids can be reacted with alkali metal hydroxides, amines and metals. These reactions produce salts. These reactions also produce hydrogen or water. The reaction will produce nitrogen gas, which is toxic in large volumes, however, only a minimal volume of nitrogen gas would be produced. Carboxylic acids can also be reacted with hydrides to make a salt, which then can be made into an aldehyde. However, with a strong hydride, they can be made into alcohols.

Enzymes can also be used to convert carboxylic acids into aldehydes and alcohols. Many hydrides can also be used to convert carboxylic acids into aldehydes and alcohols. The aldehyde of the main acid in the reaction is not toxic and has a smell that is not unpleasant. Metal carbonates can also be used to make salts. Sodium carbonate and silver carbonate can be used. Some can be irritating and the metals with pleasant-smelling salts should be used. However, most salts do not have pleasant smells. Sodium borohydride can also be used to make salts of carboxylic acids. Bases also react to make salts and water, so bases that are not irritating can be used. Anion exchange resins can also be used. Diaion products are resins that can be used. Diaion™ PA 412 in the —OH (hydroxyl) can be used because it will produce a salt at the temperatures similar to the temperature of the skin area to which the composition will be applied. Since these reactions will produce water, silica gel or hydro gel can be used. However, the water should not react with the salt to make the acid. This reaction may not be optimal because some organic acid salts do not have pleasant smells. However, the organic acid salts do not have a strong unpleasant smell, so this can be used to make the unpleasant smell less strong.

Heteropoly acids are another exemplary class of acids that can also be used.

As noted, bacteria lives in perspiration generated by the body and such bacteria generate acids that often lead to foul or unpleasant body odor. The embodiments discussed herein include compositions that react with the acids formed by the bacteria and which, in turn, undergo a reaction that generates a pleasant-smelling ester, which, in turn, eliminates, reduces, or masks negative body odor.

Deodorants and antiperspirants typically contain water, solubilizer, emollient, deodorant active ingredients, ethanol, and preservatives. The main ingredient of these deodorants are the deodorant active ingredients. Oher ingredients include thickeners and carriers for the deodorant active ingredients. The ingredients used to thicken are waxes and starches. The carriers are ethanol, water, propylene glycol and glycerol. The deodorants also have ingredients to give the deodorant a pleasant smell. The alcohols that will be used have a pleasant smell. Since the alcohols will make the deodorant have a pleasant odor, chemicals that deodorants typically use for smell do not need to be present in the deodorant because they will decrease the concentration of the alcohols. Alcohols can be used as a solvent and an ingredient to make the composition thicker, so this will increase the concentration of alcohols that can react with the acid and produce an ester. The instant composition can be used with deodorant active ingredients and antiperspirant technologies to increase the effectiveness of the deodorant. The deodorant compositions and mixtures of these ingredients can be used in some present commercial embodiments. The deodorants typically use a percent of weight for the deodorant active ingredient of 0 to 11. The antiperspirants typically use a percent of weight for the deodorant active of 1 to 20.

The instant disclosure is directed to a composition that comprises an alcohol, typically having 1-29 carbons, one or more catalysts, which may include sulphonated compounds, weak acids, strong cation exchange resins, enzymes, metal oxides and organic catalysts.

A deodorant that masks body odor or negative odor from perspiration can be formulated with alcohols and a catalyst. Alcohols having many hydroxy groups are preferred. For a composition of a base that is used to react with the isovaleric acid and creates a salt, there should be 30 to 90 percent by weight of the base.

Deodorants may often include a small amount of alcohol. In this manner, the instant composition provides a catalyst to generate a reaction of the alcohol with acids generated by bacteria in perspiration such that the catalyst will reduce the amount of acid and generate a pleasant-smelling ester.

As detailed herein, there are several methods of causing the alcohol to react with the acids in perspiration to create an ester that reduces/eliminates body odor or generates a pleasant-smelling odor.

If an ester is applied with a strong cation exchange resin, the ester will react with the water. The strong cation exchange resin will catalyze the reaction. The area will be dry, which is good. The ester will produce an alcohol and an acid, the acid that is used should not have an unpleasant smell. The number of carbons should be high for the ester that makes the acids. The acids have a higher number of carbons, so the unpleasant smelling acids, which are small, will react to make an ester.

Another method for preparing the composition is to use an ester and a lipase. The lipase can catalyze the reaction of the ester and water. The lipase can also catalyze the reaction of creating the ester. An enzyme and catalyst can be used.

An alkene can be used to create an alcohol, which can make an ester. The alkene will use a hydratase enzyme to react with water to create the alcohol. This will make the area dry. There should also be a catalyst for the reaction of creating the ester. A salt is made by reacting an acid and a base, which makes a salt of the acid and the metal from the base. If there is sodium hydroxide that is used, it will make a sodium salt, if a salt of a weaker acid is used, it will react with the unpleasant smelling acid and create a salt, which has a smell that is not as strong as the acid.

When an alcohol is protonated by a strong acid it creates an oxonium ion (protonated alcohol), which can create an ester if it reacts with an acid. The ion can be the functional group of a resin. This will create the ester efficiently. The alcohols with the amount of carbons noted in this disclosure can be used.

The oxonium ion functionalized resin can be prepared by adding a large amount of strong acid, such as sulfuric acid, to an alcohol of the number of carbons noted herein. The strong acid will protonate the alcohol. The protonated alcohol should then be purified. Many methods can be used, such as filtration. The protonated alcohol should then be added as the functional group of resin or polystyrene. Many resins and polymers can be used. These catalysts may comprise beads or they can be ground up.

Alkynes can make esters and can also be used. The alkynes are in the carbon range of the alcohols.

Alkenes may also be used. The alkenes should have 1 to 29 carbons.

As noted, the composition of the instant disclosure can be used as a solid stick, roll-on or a spray or wet wipe. The stick will include one or more ingredients to make it thick and easy to use. The spray will typically include a propellant or aerosol and include a volatile chemical to make it effective. The deodorant stick composition can also use a volatile chemical to make the deodorant dry.

Catalysts are useful in the instant composition to facilitate or expedite the reactions. Useful catalysts include, but are not limited to, acidic aluminosilicates, sulfonated objects, sulfonated oilseed cake, sulfonated silica. silica, alumina catalyst, sulfate supported metal oxides, iron oxide supported on SBA 15, Iron oxide on many mesoporous materials, Nano catalysts using UiO, sulfonic acid with functionalized metal framework ZrO₂, sulfonated Al₂O₅, Amberlyst® 15, Amberlyst® 35, Amberlyst™ 39, and Dowex H+ resins, solid acid catalysts, SBA 15, iron oxide on many mesoporous materials, nano catalysts using UiO, sulfonic acid with functionalized metal framework, MOF with SO₃H, AL₂O₃, RF resin, metal salts, Zr, Al and Si.

In a first embodiment, a composition for generating a pleasant smell in a deodorant or antiperspirant product comprises an alcohol, a fast evaporating chemical, thickener, a catalyst, and silica.

In the first embodiment, alcohols with 1-29 carbons can be used. More preferably, alcohols with 3-19 carbons can be used. Chemicals such as terpenes, terpenoids or sesquiterpene with one or more hydroxy groups may also be used, as well as other compounds with hydroxy groups. Alkynes can also be used. Alcohols serves as the reactant in the esterification reaction of the instant disclosure. Typically, a larger amount of alcohol will produce a larger yield of the desired product. In this first embodiment alcohols will be present in the range of 0.1 to 100% by weight. More preferred, the alcohols will be present in the range of 60 to 90% by weight. Even more preferred, the alcohols will be present in the range of 70 to 80% by weight.

In the first embodiment, a solvent should be used. Volatile solvents such as silicones can be used. Silicones, such as for example, siloxanes and cyclomethicone are suitable for deodorant sticks since they evaporate quickly and thus are not wet. Siloxanes and cyclomethicone are silicones that are commonly used in deodorant products as a liquid carrier. These silicones are typically volatile and facilitate application of the deodorant product because they evaporate quickly. Solvents do not need to be volatile and many other solvents can be used. In certain embodiments, polyhydric alcohols may also be used since the polyhydric alcohols react with an added catalyst to create esters with a pleasant smell. In the first embodiment, the solvent will typically be in the range of 0 to 50% by weight. Even better, in the range of 0 to 40% by weight, and most preferred in the range of 0 to 30% by weight.

The composition of the first embodiment may optionally include additional chemical compounds or ingredients that are used to thicken the deodorant product and render it easier to apply. Such thickening agents typically may comprise fatty alcohols and amides. For example, paraffin and hydrogenated triglycerides are typically added to the composition. When fatty alcohols are used, they create weak smelling or pleasant-smelling esters. Preferred fatty alcohols in the composition of the first embodiment typically have 8 to 50 carbons. The thickening agents will be present in the amount of 0 to 40% by weight. In a more preferred embodiment, the thickening agent will be in the range of 0 to 30% by, and in even more preferred in the range of 0 to 20% by weight.

The composition of the first embodiment will also include a catalyst. Exemplary catalysts include, but are not limited to, acidic aluminosilicates, sulfonated objects, sulfonated oilseed cake, sulfonated silica. silica, alumina catalyst, sulfate supported metal oxides, ZrO₂, sulfonated Al₂O₅, Amberlyst® 15, Amberlyst® 35, Amberlys™t 39, and Dowex H+resins, solid acid catalysts. SBA 15, iron oxide on many mesoporous materials, nano-catalysts using UiO, sulfonic acid with functionalized metal framework, MOF with SO₃H, AL₂O₃, RF resin, metal salts, Zr, Al and Si. The catalyst will exist in the range of 0 to 90% by weight. More preferred, the catalyst will be in the range of 0 to 70% by weight and even more preferred in the range of 0 to 50% by weight.

Lipases or enzymes come from bacteria, e.g. Candida rugosa, Candida antarctica, Aspergillus niger, Aspergillus oryzae, Thermomyces lanuginosus, and Rhizomucor miehei, and acyltransferases may also serve as catalysts.

The composition of the first embodiment may further include silica or molecular sieves. Silica or molecular sieves will typically exist in the range of 0 to 60% by weight. More preferred, in the range of 8 to 50% by weight, and even more preferred in the range of 10 to 30% by weight.

In a second embodiment, a composition for generating a pleasant smell in a deodorant or antiperspirant is when the composition comprises fatty alcohols as thickener that do not generate a smell or odor.

The second embodiment comprises alcohols with 1-29 carbons. More preferably, alcohols with 3-19 carbons can be used. Chemicals such as terpenes, terpenoids or sequiterpene with one or more alcohol groups may also be used, as well as other compounds with hydroxy groups. Alcohols in the second embodiment will typically be in the range of 0.1 to 90%. More preferred in the range of 10 to 50%, and even more preferred in the range of 20 to 40%.

The second embodiment composition comprises silicones or other solvents that serve as drying agents or chemicals that evaporate quickly. Siloxanes and cyclomethicone are suitable for deodorant sticks since they evaporate quickly and thus are typically not wet. The solvents typically exist in the range of 0 to 60%. More preferred in the range of 10 to 50% by weight, and even more preferred in the range of 20 to 40% by weight.

The second embodiment also includes a fatty alcohol with 8 to 50 carbons as a thickener. Preferred fatty alcohols have 8 to 50 carbons. Even more preferred, the fatty alcohols have 11-19 carbons. Fatty acids in this composition typically result in a thick deodorant formulation. The size of the alcohol is related to the yield of testers produced in the reaction and as a result, to the smell produced by the esters. The fatty alcohols disclosed in this embodiment will typically make esters with little to no smell. Alcohols with more carbons will make esters with even less smell. Smaller alcohols will yield esters with more smell. The smell of the esters of the alcohols will yield either no smell or pleasant smells for incorporation into the deodorant product. The fatty alcohol will typically in the range of 0 to 90%. More preferred in the range of 10 to 50% by weight ,and even more preferred in the range of 20 to 40% by weight.

The composition of the second embodiment will also include a catalyst. Exemplary catalysts include, but are not limited to, acidic alumino silicates, sulfonated objects, sulfonated oilseed cake, sulfonated silica. silica, alumina catalyst, sulfate supported metal oxides, ZrO2, sulfonated A1205, Amberlyst® 15, Amberlyst® 35, Amberlyst™ 39, and Dowex H+resins, solid acid catalysts. SBA 15, iron oxide on many mesoporous materials, nano catalysts using UiO, sulfonic acid with functionalized metal framework, MOF with SO₃H, AL₂O₃, RF resin, metal salts, Zr, Al and Si. The catalyst will typically be present in the range of 0 to 70% by weight. More preferred in the range of 8 to 50%, and even more preferred in the range of 10 to 40%.

The second embodiment optionally includes silica, which will typically be in the range of 0 to 60% by weight. More preferred in the range of 8 to 50%, and even more preferred in the range of 10 to 30%.

A third embodiment of the composition comprises alcohols with 1-29 carbons. More preferably, alcohols with 3-19 carbons can be used. Chemicals such as terpenes, terpenoids or sesquiterpene with one or more alcohol groups may also be used, as well as other compounds with hydroxy groups. The alcohols are present in the range of 0.1 to 90% by weight. More preferred in the range of 10 to 50%, and even more preferred in the range of 20 to 40%.

The third embodiment of the composition also includes polyhydric alcohols with 1 to 40 hydroxy groups.

Advantageously, the polyhydric alcohols include 1 to 40 hydroxy groups, more preferably with 2 to 9 hydroxy groups, and even more preferably with 2 to 4 hydroxy groups. The polyhydric alcohol will be present in the range of 0 to 60% by weight. More preferred in the range of 10 to 50% by weight, and even more preferred in the range of 20 to 40% by weight.

The third embodiment of the composition also includes waxes and fatty amides, including but not limited to, hydrogenated castor, parafin wax, bees wax, carnauba, ceresin and microcrystallines, and polyethylene waxes. The waxes exist in the range of 0 to 90% by weight. More preferred in the range of 10 to 50% by weight, and even more preferred in the range of 20 to 40% by weight.

The composition of the second embodiment will also include a catalyst. Exemplary catalysts include, but are not limited to, acidic alumino silicates, sulfonated objects, sulfonated oilseed cake, sulfonated silica. silica, alumina catalyst, sulfate supported metal oxides, ZrO₂, sulfonated A1205, Amberlyst® 15, Amberlyst® 35, Amberlyst 39™, and Dowex H+ resins, solid acid catalysts. SBA 15, iron oxide on many mesoporous materials, nano catalysts using UiO, sulfonic acid with functionalized metal framework, MOF with SO₃H, AL₂O₃, and RF resin, metal salts, Zr, Al and Si. The catalyst will typically be present in the range of 0 to 90% by weight. More preferred in the range of 8 to 50% by weight, and even more preferred in the range of 20 to 40% by weight.

The third embodiment of the composition further includes silica in the range of 0 to 60% by weight. More preferred in the range of 8 to 50% by weight, and even more preferred in the range of 10 to 30% by weight.

The composition of the third embodiment will produce lower yield of esters with a great deal of smell or odor. The deodorant composition in this embodiment has a larger amount of alcohols that generate esters that do not have substantial smell.

A fourth embodiment of the composition comprises alcohols with 1-29 carbons. More preferably, alcohols with 3-19 carbons can be used. Chemicals such as terpenes, terpenoids or sesquiterpene with one or more alcohol groups may also be used, as well as other compounds with hydroxy groups. The alcohols are present in the range of 0.1 to 90% by weight. More preferred in the range of 10 to 50%, and even more preferred in the range of 20 to 40%.

The fourth embodiment of the composition also includes polyhydric alcohols with 1 to 40 hydroxy groups. In a preferred embodiment the polyhydric alcohol contains 2 to 9 hydroxy groups, and in a more preferred embodiment the polyhydric alcohol contains 2 to 4 hydroxy groups. The polyhydric alcohol will be present in the range of 0 to 90% by weight. More preferred in the range of 10 to 50%, and even more preferred in the range of 20 to 40%.

The fourth embodiment further includes a fatty alcohol with 8 to 50 carbons. In a preferred embodiment the fatty alcohol contains 8-50 carbons, and in a more preferred embodiment the fatty alcohol contains 11 to 19 carbons.

The composition of the fourth embodiment will also include a catalyst. Exemplary catalysts include, but are not limited to, acidic alumino silicates, sulfonated objects, sulfonated oilseed cake, sulfonated silica. silica, alumina catalyst, sulfate supported metal oxides, ZrO₂, sulfonated Al₂O₅, Amberlyst® 15, Amberlyst® 35, Amberlyst 39™, and dowex H+ resins, solid acid catalysts. SBA 15, iron oxide on many mesoporous materials, nanocatalysts using UiO, sulfonic acid with functionalized metal framework, MOF with SO₃H, AL₂O₃, RF resin, metal salts, Zr, Al and Si. The catalyst will typically be present in the ranger of 0 to 80% by weight. More preferred in the ranger of 8 to 50% by weight, and even more preferred in the range of 20 to 40% by weight.

The fourth embodiment of the composition further includes silica in the range of 0 to 60% by weight. More preferred in the range of 8 to 50% by weight, and even more preferred in the range of 10 to 30% by weight.

A fifth embodiment comprises an ester of an alcohol with 1 to 29 carbons The ester is an acid with 12 to 34 carbons. This ester can be with a polycarboxylic acid with 1 to 40 carbons. The ester of alcohol will typically be in the range of 0.1 to 90% by weight. More preferred, in the range of 40 to 80% by weight, and even more preferred in the range of 50 to 70% by weight.

The composition of the fifth embodiment uses less water to enable a dryer area so that the ester reacts with the water present to produce an acid and an alcohol. The acid is one that is has a larger number of carbons than the malodorous acid such that the result will be an ester and acid devoid of smell.

The fifth embodiment further comprises a catalyst. Exemplary catalysts include, but are not limited to, acidic alumino silicates, sulfonated objects, sulfonated oilseed cake, sulfonated silica. silica, alumina catalyst, sulfate supported metal oxides, ZrO₂, sulfonated Al₂O₅, Amberlyst® 15, Amberlyst® 35, Amberlyst 39™, and dowex H+ resins, solid acid catalysts. SBA 15, iron oxide on many mesoporous materials, nano catalysts using UiO, sulfonic acid with functionalized metal framework, MOF with SO₃H, AL₂O₃, RF resin, metal salts, Zr, Al and Si. The catalyst will typically be present in the range of 0.1 to 90% by weight. More preferred in the range of 8 to 50% by weight, and even more preferred in the range of 10 to 40% by weight.

The fifth embodiment also includes silica in the range of 0 to 70% by weight. More preferred in the range of 10 to 50% by weight, and even more preferred in the range of 20 to 40% by weight.

A sixth embodiment of the instant disclosure comprises an ester of an alcohol with 1 to 29 carbons. The ester can also be with a polyhydric alcohol with 1 to 40 hydroxy groups, more preferably with 2 to 9 hydroxy groups, and even more preferably with 2 to 4 hydroxy groups. The ester is a solid. The ester can be with a carboxylic acid that has 12 to 34 carbons. This ester can be with a polycarboxylic acid with 1 to 40 carbons. In a preferred embodiment the ester is an ester of an alcohol with minimal odor or smell, such as for example, a polyhydric alcohol. The ester of an alcohol will typically be in the range of 0.1 to 100% by weight. More preferred, in the range of 20 to 90%, and even more preferred in the range of 40 to 80%.

The sixth embodiment also comprises silicones such as siloxanes and cyclomethicone. The silicones will be in the range of 0 to 60% by weight. More preferred, in the range of 8 to 50% by weight, and even more preferred in the range of 10 to 40% by weight.

The sixth embodiment also comprises a weak base, for example, metal carbonate, metal hydroxide or salt. The weak base will typically be in the range of 0 to 60% by weight. More preferred in the range of 10 to 50% by weight, and even more preferred in the range of 20 to 40% by weight.

The sixth embodiment further includes silica in the range of 0 to 70% by weight. More preferred in the range of 10 to 80% by weight, and even more preferred in the range of 20 to 50% by weight.

The compositions of the embodiments disclosed herein may optionally include an ester.

A seventh embodiment of the composition of the instant disclosure includes an ester in the amount of 20 to 100% by weight. More preferred, the ester is present in the amount of 40 to 80% by weight. Even more preferred, the ester is present in the amount of 50 to 60% by weight.

The composition of the seventh embodiment will also include a catalyst and an enzyme.

The catalyst in the seventh embodiment will be in the amount of 0 to 70% of weight. More preferably in the amount of 20 to 60% of weight, and even more preferably in the amount of 30 to 40% of weight.

The enzyme in the seventh embodiment will be in the amount of 0 to 80% of weight. More preferably in the amount of 10 to 50% of weight, and even more preferably in the amount of 20 to 40% of weight.

In the seventh embodiment, the ester will react with water in the composition and will be catalyzed by the catalyst and optionally an enzyme. A preferred catalyst is an acid catalyst. The ester will produce an acid and an alcohol. The acid in this embodiment will be one without a strong smell and will have more carbons than any acid with a poor smell. In this manner the isovaleric acid caused by the reaction of bodily bacteria with the acid will reaction with the alcohol to create a more pleasant-smelling ester and facilitate a dry area.

The seventh embodiment may further include silicones or polyhydric alcohols in the amount of 0 to 50% by weight. More preferred, the silicones or polyhydric alcohols in the amount of 10 to 40% by weight. Even more preferred, the silicones or polyhydric alcohols in the amount of 20 to 30% by weight.

The seventh embodiment may also include fatty alcohols in the amount of 0 to 70% by weight. More preferred, the fatty alcohols in the amount of 10 to 50% by weight. Even more preferred, the fatty alcohols in the amount of 20 to 40% by weight.

In an eighth embodiment, the composition of the instant disclosure will include an ester, a catalyst, and an enzyme.

The ester in the eighth embodiment will be an alcohol that has 1 to 29 carbons and an acid that has 8 to 50 carbons. The alcohol can be a polyhydric alcohol. The acid can be a polycarboxylic acid. The ester should be of an alcohol with one hydroxy group and a polycarboxylic acid.

The eighth embodiment will include ester in the amount of 20 to 100% by weight. More preferred in the amount of 40 to 80% by weight, and even more preferred in the amount of 50 to 60% by weight.

The catalyst in the eighth embodiment will be in the amount of 0 to 80% by weight. More preferred, the amount of catalyst will be in the amount of 10 to 50%. Even more preferred, the catalyst will be in the amount of 30 to 40%.

The preferred alcohol in the eighth embodiment will have 1 to 20 carbons and will be in the amount of 0 to 70%. More preferred, the alcohol will be in the amount of 10 to 40%. Even more preferred, the alcohol will be in the amount of 20 to 30%.

The eighth embodiment may further include silicones or polyhydric alcohols in the amount of 0 to 50% by weight. More preferred, the silicones or polyhydric alcohols in the amount of 10 to 40% by weight. Even more preferred, the silicones or polyhydric alcohols in the amount of 20 to 30% by weight.

The eighth embodiment may also include fatty alcohols in the amount of 0 to 70% by weight. More preferred, the fatty alcohols in the amount of 10 to 50% by weight. Even more preferred, the fatty alcohols in the amount of 20 to 40% by weight.

In a ninth embodiment, the composition of the instant disclosure will include an alkene and a catalyst. The alkene will react with the water to create an alcohol and can be catalyzed by an acid catalyst. The catalyst will also catalyze the reaction of the ester with the acid.

A preferred alkene in the ninth embodiment will have 1 to 20 carbons and will be in the amount of 20 to 100% of weight. More preferred, the alkene will be in the amount of 40 to 80% of weight. Even more preferred, the alkene will be in the amount of 50 to 70% of weight

A preferred catalyst in the ninth embodiment will be an acid or sulfonic group functionalized resin and will be in the amount of 0 to 70% of weight. More preferred, the catalyst will be in the amount of 10 to 50% of weight. Even more preferred, the catalyst will be in the amount of 20 to 40% of weight.

The ninth embodiment may further include silicones or polyhydric alcohols in the amount of 0 to 50% by weight. More preferred, the silicones or polyhydric alcohols in the amount of 10 to 40% by weight. Even more preferred, the silicones or polyhydric alcohols in the amount of 20 to 30% by weight.

The ninth embodiment may also include fatty alcohols in the amount of 0 to 70% by weight. More preferred, the fatty alcohols in the amount of 10 to 50% by weight. Even more preferred, the fatty alcohols in the amount of 20 to 40% by weight.

In a tenth embodiment, the composition of the instant disclosure will include an alkene, a hydratase enzyme and an additional enzyme that will catalyze the reaction of an acid and alcohols.

A preferred alkene of the tenth embodiment will have 1 to 20 carbons and will be in the amount of 0 to 80% of weight. More preferred, the alkene will be in the amount of 30 to 60% by weight, and even more preferred in the amount of 40 to 60% by weight.

The hydratase enzyme of the tenth embodiment which catalyzes the reaction of the alkene and water which creates an alcohol will be in the range of 0 to 50% by weight. More preferably the hydratase enzyme will be in the range of 10 to 40%, and even more preferably in the amount of 20 to 40% by weight. Exemplary hydratase enzymes include, but are not limited to, oleate hydratase, linalool hydratase and fatty acid hydratase. An additional enzyme is there to catalyze the reaction of that produced alcohol together with the unpleasant smelling acid and to produce an ester. This enzyme can be a lipase. This additional enzyme of the tenth embodiment will be in the range of 0 to 70% by weight. More preferably that enzyme will be in the range of 10 to 50%, even more preferably in the range of 20 to 40% by weight.

It is noted that for ingredients such as linalool, said ingredients should be mixed in an area with no oxygen because they can form unwanted chemicals that will not react as well.

The tenth embodiment may further include silicones or polyhydric alcohols in the amount of 0 to 50% by weight. More preferred, the silicones or polyhydric alcohols in the amount of 10 to 40% by weight. Even more preferred, the silicones or polyhydric alcohols in the amount of 20 to 30% by weight.

The tenth embodiment may also include fatty alcohols in the amount of 0 to 70% by weight. More preferred, the fatty alcohols in the amount of 10 to 50% by weight. Even more preferred, the fatty alcohols in the amount of 20 to 40% by weight.

In an eleventh embodiment, the composition will include resin with alkoxonium ion of alcohol having 1 to 20 carbons. A preferred resin is polystyrene or a polymer with an oxonium ion as the group. The resin will be in the amount of 10 to 20% by weight. More preferred, the resin will be in the amount of 20 to 90% by weight, and even more preferably, the resin will be in the amount of 50 to 80% by weight.

In a twelfth embodiment, a molecular sieve 13X is utilized, with 0 to 90% of weight. More preferably 20 to 80% by weight, and even more preferably 30 to 60% by weight

This is a type of molecular sieve that was tested and will dry the isovaleric acid and make the smell less strong. The molecules in the sieve should be ground up.

The twelfth embodiment will also include alcohol. Preferably a polyhydric alcohol with 1-40 hydroxy groups and in range of 20 to 90%of weight. More preferably in the range of 30 to 80% by weight and even more preferably in the range of 40 to 60%by weight. These can also be used, ethanol and water.

A wax component can also be incorporated in the composition of the twelfth embodiment. The wax will be in the range of 10 to 90%by weight. More preferably the wax will be in the range of 30 to 80% by weight, and even more preferably in the range of 40 to 60% by weight.

In yet another embodiment, embodiment 13, the composition incorporates deodorant or antiperspirant active ingredients in the amount of 0 to 80% by weight. Preferably in the amount of 10 to 50% by weight, and more preferably in the amount of 20 to 50% by weight.

This embodiment also includes polyhydric alcohols with 1 to 40 hydroxy groups in the amount of 0 to 90% by weight. Preferably the polyhydric alcohol is in the amount of 20 to 80% by weight, and even more preferably in the amount of 50 to 60% by weight. In addition, ethanol and water can be used as solvents.

A wax component can also be incorporated in the composition of the thirteenth embodiment. The wax will be in the range of 0 to 80% by weight. More preferably the wax will be in the range of 10 to 50% by weight, and even more preferably in the range of 30 to 40% by weight.

The following composition in the Examples correspond with embodiments as disclosed in the Detailed Description hereinabove.

### EXAMPLES

Composition Embodiment 1

| Ingredient | % w/w |
|---|---|
| Alcohols with 1 to 29 carbons | 70 to 80 |
| Silicones | 0 to 30 |
| Fatty alcohols with 8 to 50 carbons | 0 to 20 |
| Catalyst | 0 to 50 |
| Silica | 10 to 30 |

Composition Embodiment 2

| Ingredient | % w/w |
|---|---|
| Alcohols with 1 to 29 carbons | 20 to 40 |
| Silicones | 20 to 40 |
| Fatty alcohols with 8-50 carbons | 20 to 40 |
| Catalyst | 10 to 40 |
| Silica | 10 to 30 |

Composition Embodiment 3

| Ingredient | % w/w |
|---|---|
| Alcohols with 1 to 29 carbons | 20 to 40 |
| Polyhydric Alcohols with 1 to 40 hydroxy groups | 20 to 40 |
| Waxes/Fatty Amides | 20 to 40 |
| Catalyst | 20 to 40 |
| Silica | 10 to 30 |

Composition Embodiment 4

| Ingredient | % w/w |
|---|---|
| Alcohols with 1 to 29 carbons | 20 to 40 |
| Polyhydric Alcohols with 1 to 40 hydroxy groups | 20 to 40 |
| Fatty alcohols with 8-50 carbons | 20 to 40 |
| Catalyst | 10 to 40 |
| Silica | 10 to 30 |

Composition Embodiment 5

| Ingredient | % w/w |
|---|---|
| Ester of an alcohol with 1 to 29 carbons and an acid | 50 to 70 |
| with 12 to 34 carbons | |
| Catalyst | 10 to 40 |
| Silica | 20 to 40 |

Composition Embodiment 6

| Ingredient | % w/w |
|---|---|
| Ester of an alcohol with 1 to 29 carbons and an | 40 to 80 |
| Acid with 12 to 34 carbons | |
| Silicones | 10 to 40 |
| Weak base | 20 to 40 |
| Silica | 20 to 50 |

Composition Embodiment 7

| Ingredient | % w/w |
|---|---|
| Ester | 50 to 60 |
| Catalyst | 30 to 40 |
| Enzyme | 20 to 40 |

Composition Embodiment 8

| Ingredient | % w/w |
|---|---|
| Ester | 50 to 60 |
| Catalyst | 30 to 40 |
| Alcohol | 20 to 30 |

Composition Embodiment 9

| Ingredient | % w/w |
|---|---|
| Alkene | 50 to 70 |
| Catalyst | 20 to 40 |

Composition Embodiment 10

| Ingredient | % w/w |
|---|---|
| Alkene | 40 to 60 |
| Hydratase enzyme | 20 to 30 |
| Enzyme | 20 to 40 |

Composition Embodiment 11

| Ingredient | % w/w |
|---|---|
| Resin with alkoxonium ion of | 50 to 80 |
| an alcohol with 1-20 Carbons | |

Composition Embodiment 12

| Ingredient | % w/w |
|---|---|
| Molecular sieve, 13X | 40 to 60 |
| Polyhydric Alcohols with 1 to 40 hydroxy groups | 40 to 60 |
| Wax | 40 to 60 |

Composition Embodiment 13

| Ingredient | % w/w |
|---|---|
| Deodorant/Antiperspirant actives | 20 to 40 |
| Polyhydric Alcohols with 1 to 40 hydroxy groups | 50 to 60 |
| Wax | 30 to 40 |

The instant disclosure contemplates that the compositions of the embodiments detailed above can be mixed with each other without detracting from the intended effect of creating a pleasant smell.

Method of Making the Deodorant Composition

The deodorant compositions can be made by mixing the liquid ingredients of the compositions and then adding the wax. The wax can be added at a high temperature so that the wax is a liquid in order to make the deodorant into a solid.

Method of Making a Spray Deodorant

The spray compositions can be made by mixing ingredients of the compositions as set forth above and pressurized for containment within a can.

As stated above, mixtures of the compositions can be used, or the compositions can be used with deodorant active ingredients. The compositions can also be used with antiperspirant active ingredients. In these embodiments, a smaller amount of the chemicals would be used, and a larger amount of the deodorant active ingredients would be used.

Method of Making a Bacterial Composition

A bacteria composition can be made by drying the bacteria and suspending them in a propellant, a low boiling point hydrocarbon. This would be for a spray. For a stick, the bacteria would be added to a liquid carrier that would evaporate quickly, a volatile silicone. In these compositions, the bacteria would act as a catalyst when there is water in the area. This is when there would be sweat. There would also be isovaleric acid. The bacteria would then produce the esters. The alcohol that is used as the ester producing alcohol should be of a large amount so that it does not kill bacteria.

Method of Making an Alternative Bacteria Composition

For making bacteria wipes, wipes can put into a culture of a suitable bacteria with added starches from food. The wipe would then be applied to the area of the subject's body. The alcohol would be present on the wipes and the alcohol that is used should not kill bacteria.

Method for Testing the Compositions

The compositions were tested by mixing them in a beaker and slowly adding 0.1 ml of concentrated isovaleric acid. The isovaleric acid was added carefully so no acid was on the walls of the beaker. This would make unreacted isovaleric acid present and the results would not be accurate. This experiment was done at room temperature. The results were rated by two people who used a 10-point scale. These people were not around the samples for an amount of time that made them not accustomed to the smell of the acid. The samples were smelled every ten minutes for about an hour. The samples were mixed before they were smelled so that all of the chemicals in the sample could be smelled. The samples were smelled and rated. There was some stirring while the samples were reacting. The samples were then poured on a paper towel to rate the strength of the smells.

When introducing elements or aspects of the invention or the embodiments thereof, the articles “a,” “an,” “the,” and “said” are intended to mean that there are one or more of the elements, and thus may include plural referents unless the context clearly dictates otherwise. The terms “comprising,” “including,” and “having” are intended to be inclusive and mean that there may be additional elements other than those listed elements.

Having described aspects of the invention in detail, it will be apparent that modifications and variations are possible without departing from the scope of aspects of the invention as defined in the appended claims. As various changes could be made in the above compositions, products, and methods without departing from the scope of aspects of the invention, it is intended that all matter contained in the above description shall be interpreted as illustrative and not in a limiting sense.

While the foregoing description refers to particular illustrative embodiments, these examples should not be construed as limitations. The inventive system, methods, and products can be adapted for other uses or provided in other forms not explicitly listed above and can be modified in numerous ways within the spirit of the present disclosure. Thus, the present invention is not limited to the disclosed embodiments but is to be accorded the widest scope consistent with the claims below.

What is claimed is:

1. A composition for initiating a reaction between a catalyst and acid present in perspiration generated on the body of a human subject, the composition comprising:
a. an alcohol;
b. a solvent;
c. a thickener;
d. a catalyst;
e. silica; and
f. a polyhydric alcohol,
wherein the composition catalyst will catalyze the reaction of the acid and the alcohol such that the reaction generates an ester having a pleasant scent.

2. The composition according to claim 1, wherein said alcohols have 1 to 29 carbons.

3. The composition according to claim 1 wherein the silicone compound is selected from the group consisting of siloxanes and cyclomethicones.

4. The composition according to claim 1, wherein the fatty alcohols have 8 to 50 carbons.

5. The composition according to claim 1, wherein the catalyst is selected from the group consisting of acidic aluminosilicates, sulfonated objects, sulfonated oilseed cake, sulfonated silica, silica, alumina catalyst, sulfate supported metal oxides, ZrO₂, sulfonated Al₂O₅, Amberlyst® 15, Amberlyst® 35, Amberlyst 39™, and dowex H+ resins, solid acid catalysts. SBA 15, iron oxide on many mesoporos materials, nano catalysts using UiO, sulfonic acid with functionalized metal framework, MOF with SO₃H, AL₂O₃, RF resin, metal salts, Zr, Al, Si, lipases, enzymes and acyltransferases, oleate hydratase, linalool hydratase and fatty acid hydratase.

6. The composition according to claim 1, wherein the catalyst comprises one or more bacteria.

7. The composition according to claim 1, wherein the solvent comprises a silicone compound

8. The composition according to claim 1, wherein the thickener comprises a fatty alcohol.

9. The composition according to claim 1, further comprising an ester.

10. The composition according to claim 9, further comprising an alkene, wherein the composition will catalyze the reaction of acid present on the subject's body and said alcohol, ester or alkene, and wherein the reaction of said acid with said alcohol, the ester or the alkene results in a pleasant odor or minimal odor.

11. The composition according to claim 10, wherein the alkene comprises 1 to 29 carbons

12. The composition according to claim 9, wherein said ester comprises at least one alcohol of 1 to 29 carbons or a polyhydric alcohol of 1 to 40 hydroxy groups and an acid of 12 to 34 carbons.

13. The composition according to claim 1, wherein the catalyst comprises a source of heat or electricity.

14. The composition according to claim 1, further comprising an oxonium ion functionalized resin.

15. The composition according to claim 14, wherein the oxonium ion comprises an alcohol with 1 to 29 carbons.

16. A composition for creating a reaction of a base and an acid to produce the salt of the acid, wherein the salt has less odor than the acid, and can be incorporated into a deodorant composition.

17. The composition according to claim 16, wherein the base can be selected from the group consisting of a metal carbonate, metal oxide or salt.

18. The composition according to claim 16, further comprising a catalyst, wherein the catalyst comprises a source of heat or electricity.

19. A method of improving the scent of body odor and perspiration on the body of a user, comprising the steps of:
a. preparing a personal care composition in a suitable carrier for application to the skin of a human subject, the composition comprising alcohol, a silicone compound, fatty alcohol, a catalyst, and silica;
b. packaging the personal care composition; and
c. applying the personal care composition to a portion of a human subject's body commonly associated with unwanted body odor and perspiration to reduce body odor,

20. The method according to claim 19, wherein the composition initiates a reaction between the catalyst and acid present in perspiration generated on the body of a human subject, and wherein the composition catalyst will catalyze the reaction of the acid and the alcohol such that the reaction generates an ester having a pleasant scent.
